(19) United States
(12) Patent Application Publication
(10) Pub. No.: US 2001/0051749 A1
(43) Pub. Date: Dec. 13, 2001

(54) Reaction for etherification of an aminophenol using a phase-transfer system

(76) Inventors: Jean-Roger Desmurs, St. Symphorien D'ozon (FR); Jean-Francis Spindler, Lyon (FR); Harivelo Gerard Rajoharison, Vernaison (FR)

Correspondence Address: RHODIA INC., CN-7500, 259 Prospect Plains Road, CRANBURY, NJ, 08512, US

(21) Appl. No.: 09/809,694

(22) Filed: Mar. 15, 2001

Related U.S. Application Data
(63) Continuation of application No. 09/446,655, filed on Dec. 22, 1999, now abandoned.

(30) Foreign Application Priority Data
Jun. 24, 1997 (FR) 97/07871

(51) Int. Cl. C07C 211/43

(52) U.S. Cl. 564/305

(57) ABSTRACT

The invention concerns a method for transforming an aminophenol, characterized in that it consists in a step whereby said aminophenol aniline function is in the form of an anilide function and said phenol is dissolved in a hydophobic and weakly polar solvent in the presence of water, an alkaline hydroxide, a phase transfer catalyst and a halide or an alkyl pseudo-halide. The invention is applicable to organic synthesis.

The present invention relates to a reaction for etherification of an aminophenol using a phase-transfer system. It more particularly relates to a technique for etherification of an aminophenol in which the aniline group is in the form of an anilide.

The reactions for etherification of aminophenols are generally carried out in a basic medium and using expensive solvents such as so-called polar aprotic solvents. Furthermore, this basic medium weakens the molecule and leads to colored or toxic impurities. When the ring is electron-rich, this leads to stringent precautions being taken to avoid contact with oxygen (air).

When it is electron-depleted, and when it carries good leaving groups such as fluorine, secondary reactions lead to the leaving group's elimination from the molecule.

The use of phase-transfer techniques is not widely known for this type of molecule, and mostly requires iodides whose cost is often prohibitive on an industrial scale, and the use of sulfonates (such as mesylate [methanesulfonate] or tosylate [toluene-sulfonate]) does not necessarily lead to savings on iodide, being more expensive. It also requires the use of a very large amount of sodium hydroxide very extensively above stoichiometry to obtain good anionization of the aminophenol.

When the phenol group is protected, the customary techniques involve liberation of the phenol before the etherification.

This is why one of the objects of the present invention is to provide a method which makes it possible to avoid all or some of the above drawbacks.

In particular, one object of the present invention is to provide a method of the above type which makes it possible to avoid extensively superstoichiometric consumption of sodium hydroxide.

Another object of the present invention is to provide a method of the above type which makes it possible to treat protected phenols directly without going through an independent step of liberating the phenol from its protection.

These objects, and others which will become apparent below, are achieved by means of a method for the conversion of an aminophenol, including a step in which said phenol is dissolved in a hydrophobic and weakly polar solvent in the presence of an alkali metal hydroxide, a phase-transfer catalyst and an alkylating agent selected from alkyl halides and pseudohalides, in which the aniline group of said aminophenol is in the form of an anilide group.

Entirely unexpectedly, this form of aniline on the one hand facilitates the reaction when it takes place using phase transfer, but furthermore the use of a strong base does not make the amide group react—it is not cleaved or alkylated.

The reaction may be carried out without excess, or with a small stoichiometric excess.

The aminophenols well suited to the present invention correspond to formula I:

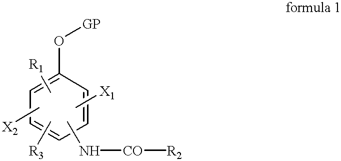

where GP represents a hydrogen or a protecting group;

where X₁ is selected from hydrogen and advantageously light halogens (chlorine or fluorine), preferably fluorine;

where X₂ and the electron-attracting groups (EAG), advantageously by inducing but not mesomeric effect; in particular they may be an alkyl which is perhalogenated (preferably perfluorinated) at least on the carbon bonded to the ring, or an advantageously light halogen (chlorine or fluorine), preferably fluorine;

where and the advantageously light (at most 6 carbon atoms) aryls and the halogens;

—CO—R₂ represents an acyl group with at most 15 carbon atoms, advantageously at most 10 carbon atoms.

Advantageously R₁ and R₃, which are similar or different, are selected from hydrogen, and alkyls with at most 4 carbon atoms.

It is desirable that, among X₁, X₂, R₁ and R₃, there is not more than three, advantageously two groups selected from the electron-attracting groups (EAG), advantageously by inducing but not mesomeric effect [alkyl which is perhalogenated (preferably perfluorinated) at least on the carbon bonded to the ring, or an advantageously light halogen (fluorine or chlorine), preferably fluorine].

The reaction can be written according to equation 1 below:

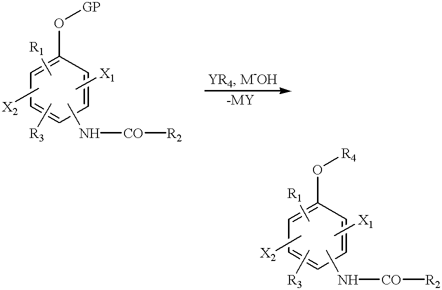

Where MOH represents an alkali metal hydroxide and R₄—Y said halide or pseudohalide of alkyl (R₄) [in the present description ALK-yl is taken in its etymological sense of a hydrocarbon residue of an ALCO-hol after ignoring the alcohol (or ol) group].

According to the present invention, it is particularly beneficial for the amount of alkali metal hydroxide to be at most equal to 2 times the SA (that is to say stoichiometric), advantageously at most equal to 1.5 times, preferably at most equal to 1.2 times.

The stoichiometric amount corresponds to the equation

Ar—OH+MOH+R₄—Y→Ar—O—R₄+MY+H₂O

When the phenol group is protected and said etherification step consists in replacing the protected phenol group by the ether group, it is desirable for the phenol group of said aminophenol to be protected by a radical which, bonded to a hydroxyl, constitutes an acid whose anionic form constitutes a leaving group. The equation then changes; in general it becomes:

Ar—OGP+2MOH+R₄—Y→Ar—O—R₄+MY+H₂O+GP—O—M

It is, however, necessary to take account of the linked reactions during the liberation of the phenol. Thus, for example, when GP corresponds to a carbonate —CO—O, the equation becomes:

Ar—O—CO—O—R₅+3MOH+R₄—Y→Ar—O—R₄+MY+H₂O+CO₃M₂+R₅—OH

It is then recommended for the phenol group of said aminophenol to be protected by a radical which, bonded to a hydroxyl, constitutes an acid whose pKa is at most equal to 6, advantageously at most equal to 5, preferably at most equal to 4.

The phenol group of said aminophenol is advantageously protected by a radical liberating an alkali metal pseudohalide, advantageously with at most 20 carbon atoms, preferably at most 10 carbon atoms.

In the present description, what is considered as a pseudohalogen is a radical [in general, this radical has a light chalcogen (sulfur or preferably oxygen) via which it is connected to the rest of the molecule] which, by leaving, constitutes an anion whose associated acid has an acidity measured by the Hammett constant at least equal to that of acetic acid. Among typical pseudohalogens, mention may be made of the acyloxyl radicals corresponding to the acids perhalogenated at alpha on the acyloxyls group, such as trifluoroacetyloxyl (CF3—CO—O—) and especially sulfonyloxyl radicals, and especially those whose carbon carrying the sulfur is perfluorinated, the paradigm of which is trifluoromethylsulfonyloxyl.

According to the present invention, also relevant are alkoxycarbonyloxyls which have an acceptable lipophilic nature and electron-attracting effect, while being less expensive.

Among the pseudohalogens, the best electron attractors are those which, by leaving, exhibit acidity at least equal to that of sulfonic acids such as tosyl (paradigm of the arylsulfonic acids) or mesylic (paradigm of the alkylsulfonic acids).

Mention should also be made of those which correspond to perfluoroalkyl sulfonic acids which have both a good electron-attracting effect and a good increase in lipophilic nature.

It is desirable for said protected phenol group to be protected by a radical liberating an alkali metal pseudohalide corresponding to protection by an acyl.

Among protecting radicals, particular mention should be made of those liberating an alkali metal pseudohalide corresponding to protection by a hydrocarbyloxycarbonyl. The carbon number of such a hydrocarbyloxycarbonyl radical is advantageously at least equal to 3, preferably at least equal to 4. It advantageously has at most 20 carbon atoms, preferably at most 10 carbon atoms.

According to an advantageous variant of the present invention, said protected phenol group is protected by a radical liberating an alkali metal pseudohalide corresponding to protection by an alkoxycarbonyl, advantageously with at most 20 carbon atoms, preferably with at most 10 carbon atoms.

The phase-transfer catalyst is selected from the following compounds: quaternary ammoniums, phosphoniums, crown ethers, cryptates and other chelating agents.

The catalyst will have to be selected so as to have extraction characteristics permitting:

extraction of the hydroxide ion from concentrated sodium hydroxide solution in order to hydrolyze the aryl carbonate to phenate.

extraction of the phenate (extraction constant between 0.1 and 1,000,000) in organic medium so as to carry out the alkylation.

The phase-transfer catalysts most suitable in industrial terms are quaternary ammoniums. The preferential substituent groups are alkyls or aryls comprising 2 to 15 carbon atoms per substituent (typically: NBu4, NBu3Bz, NEt3Bz . . . ). The counterion is either a hydroxide, halide or any other conjugate base of an acid having a pKa less than or equal to 4.

In order to obtain good results, it is desirable for the molar ratio between water and hydroxide (H₂O/OH⁻) to be at most equal to about 20, advantageously at most equal to 10, preferably at most equal to 5.

It is also recommended to carry out the addition of the reactants so that said alkyl halide or pseudohalide is, during the majority of the reaction, approximately stoichiometric or superstoichiometric with respect to that of the hydroxide or phenol which is in a limiting amount (expressed as stoichiometry).

In the same way, it is desirable for the addition of the reactants to be carried out so that the hydroxide is the limiting or colimiting reactant during the majority of the reaction.

The lipophilic medium may, in particular, be a weakly polar solvent or one of the reactants in excess, in particular said aniline, on its own or dissolved in a weakly polar solvent.

According to the present invention, it is preferable for said aniline to have a pKa at most equal to 5, advantageously at most equal to 4, preferably at most equal to 3.

The solubility in the weakly polar media also plays an important role for implementing the present invention. It is thus desirable for the solubility of said aminophenol in benzene to be at least slightly soluble (δ or d), advantageously at least soluble (s), preferably very soluble (v). The symbols are those used in the reference book Handbook of Chemistry and Physics.

It is further desirable for the hydrophobic (lipophilic) medium or solvent to be weakly miscible with water and to be hydrophobic enough not to be miscible with water in any proportion. It is thus preferable if water can dissolve only at most 10% of the solvent, or what fulfills the role of solvent; this limit is advantageously at most 5%, preferably at most 2% by mass, advantageously even in the presence of the substrate as a third solvent.

It is even preferable for the solvent to be able to dissolve only at most 10% water, advantageously at most 5%, preferably at most 2% by mass, advantageously even in the presence of the substrate as a third solvent.

These are therefore in general weakly polar solvents.

The term weakly polar solvent is intended to mean a solvent whose dielectric constant [which changes fairly little with temperature, but which is advantageously measured around 20° C., for the dielectric constant values, reference may be made to the fourth edition of the work published by John WILEY and sons “TECHNIQUES OF CHEMISTRY; Organic Solvents, Physical Properties and Methods of Purification”, by John A RIDDICK, William B. BUNGER, Theodore K. SAKANO] is at most equal to about 10 (relative dielectric constant ε). This value of ε is applicable for the main constituent of the solvent, but it is preferable for the entire solvent to satisfy this constraint.

Advantageously, the maximum value of ε is at most equal to 10 (two significant figures), preferably at most equal to 5 (the value of chlorobenzene).

According to the present invention, it is preferable for the main constituent of the solvent to be weakly basic, that is to say its donor index or donor number is at most equal to about 20 (in the present description, the term “about” is used to emphasize the fact that, when the figure or figures furthest to the right in the number are zeros, these zeros are position zeros and not significant figures, except of course if otherwise indicated), preferably at most equal to 20 (two significant figures). There is nothing critical about the lower bound.

For the definition of the donor index (donor number), reference may be made to the work by Christian Reinhardt Solvents and solvents effects in organic chemistry, p. 19 (1988), in which work the definition found is minus the enthalpy (−ΔH expressed in kilocalories/mol) of the interaction between the solvent and antimony pentachloride in a dilute dichloroethane solution.

However, when mixtures of various compounds are used as solvents, it may be beneficial for one of them, in a minor proportion, to exhibit some degree of basicity.

The solvents may be mixtures, including oil fractions. Naturally, under operating conditions, the solvents should be inert with respect to the substrates and reactants used.

The preferred families of the solvents are selected from the group consisting of hydrocarbons, aromatic derivatives, ethers, esters and halogenated solvents.

As paradigms of these families, mention may be made of the following: as halogenated aliphatic derivatives, dichloromethane, 1,2-dichloroethane, 1,1,1-trichloroethane, as aromatic derivatives, toluene, and, as halogenated aromatic derivatives, chlorobenzene, as esters, ethyl acetate and isopropyl acetate, as ethers, tert-butyl and methyl oxide as well as anisole and heavy alcohols, that is to say satisfying the immiscibility constraints as specified above.

For reasons of industrial economy, it is preferable for the solvent to be distillable under atmospheric pressure or under primary or secondary vacuum.

It is desirable for said weakly polar solvent to be selected from solvents with aromatic nature, that is to say from solvents which have at least one aromatic ring. This aromatic ring may either be present in a minor or major constituent of the solvent or, when the solvent consists of a single compound, be present in this compound (for example toluene, xylene).

The solvent should be selected so that its melting point is below the temperature at which the reaction is to take place. It is thus desirable to use weakly polar solvents from aromatic solvents which lead to a melting temperature of the reaction mixture of at most 70° C., advantageously at most 50° C.

In general, the weakly polar solvent is a solvent selected from aliphatic and/or aromatic carbides of halogenated aromatic derivatives, from esters, from phenol ethers and mixtures thereof.

Furthermore, it is advantageous to select the solvent so that its initial boiling temperature is below the boiling (or subliming) temperature of aminophenol; and if appropriate, of the other reactants employed in the method.

The organic phase is thus advantageously selected from aromatic carbides. In particular those corresponding to a substituted benzene ring and having at most about 10 carbon atoms, such as xylene, toluene, ethylbenzene and trimethylbenzene.

According to one particularly advantageous embodiment of the present invention, the reaction is carried out in the absence of iodine(ide) (that is to say at most 1 mol % with respect to the substrate, advantageously 1 per 1000, preferably 100 ppm).

The reaction is advantageously carried out at a temperature between 20 and 150° C., preferably between 30 and 120° C.

Said alkylating agent is advantageously selected from alkyl halides.

If pseudohalides are selected, it is preferable to pick those which, when leaving, have an acidity at least equal to that of sulfonic acids such as tosylic (paradigm of arylsulfonic acids) or mesylic (paradigm of alkylsulfonic acids).

Mention should also be made of those which correspond to perfluoroalkyl sulfonic acids which have both a good electron-attracting effect and a good increase in lipophilic nature.

Said alkylating agent is advantageously selected from alkyl halides. Preferably, said alkylating agent is selected from an alkyl bromide.

Said alkyl (denoted by R₄ in the formula) has from 1 to about 20 carbon atoms, preferably from 2 to about 10. The reaction gives good results for cyclic and/or secondary alkyls.

The following nonlimiting examples illustrate the invention.

1. Method for the conversion of an aminophenol, including a step in which said phenol is dissolved in a hydrophobic and weakly polar solvent in the presence of an alkali metal hydroxide, a phase-transfer catalyst and an alkylating agent selected from alkyl halides and pseudohalides, characterized in that the aniline group of said aminophenol is in the form of an anilide group.

2. Method according to claim 1, characterized in that the phenol group is protected, and in that said etherification step consists in replacing the protected phenol group by the ether group.

3. Method according to claims 1 and 2, characterized in that the phenol group of said aminophenol is protected by a radical which, bonded to a hydroxyl, constitutes an acid whose anionic form constitutes a leaving group.

4. Method according to claims 1 to 3, characterized in that the phenol group of said aminophenol is protected by a radical which, bonded to a hydroxyl, constitutes an acid whose pKa is at most equal to 6, advantageously at most equal to 5, preferably at most equal to 4.

5. Method according to claims 1 to 4, characterized in that the phenol group of said aminophenol is protected by a radical liberating an alkali metal pseudohalide.

6. Method according to claims 1 to 5, characterized in that said protected phenol group is protected by a radical liberating an alkali metal pseudohalide corresponding to protection by an acyl.

7. Method according to claims 1 to 6, characterized in that said protected phenol group is protected by a radical liberating an alkali metal pseudohalide corresponding to protection by a hydrocarbyloxycarbonyl.

8. Method according to claims 1 to 7, characterized in that said protected phenol group is protected by a radical liberating an alkali metal pseudohalide corresponding to protection by an alkoxycarbonyl, advantageously with at most 10 carbon atoms.

9. Method according to claims 1 to 8, characterized in that the molar ratio between water and hydroxide (H₂O/OH⁻) is at most equal to about 20, advantageously at most equal to 10, preferably at most equal to 5.

10. Method according to claims 1 to 9, characterized in that the addition of the reactants is carried out so that said halide or pseudohalide is stoichiometric or superstoichiometric with respect to that of the hydroxide or phenol which is in a limiting amount (expressed as stoichiometry).

11. Method according to claims 1 to 10, characterized in that the addition of the reactants is carried out so that the hydroxide is the limiting or colimiting reactant during the majority of the reaction.

12. Method according to claims 1 to 11, characterized in that the organic phase is selected from aromatic carbides.

13. Method according to claims 1 to 12, characterized in that the reaction is carried out in the absence of iodine(ide) (that is to say at most 1 mol % with respect to the substrate, advantageously 1 per 1000, preferably 100 ppm).

14. Method according to claims 1 to 13, characterized in that the reaction is carried out at a temperature between 30 and 120° C.

15. Method according to claims 1 to 14, characterized in that said alkylating agent is selected from alkyl halides.

16. Method according to claims 1 to 15, characterized in that said alkylating agent is selected from an alkyl bromide.
